(19) United States
(12) Patent Application Publication
(10) Pub. No.: US 2021/0241924 A1
(43) Pub. Date: Aug. 5, 2021

(54) METHOD FOR SUICIDE IDENTIFICATION

(71) Applicant: UNIVERSITA' DEGLI STUDI DI PADOVA, Padova (IT)

(72) Inventor: Massimo Montisci, Padova (IT)

(73) Assignee: UNIVERSITA' DEGLI STUDI DI PADOVA, Padova (IT)

(21) Appl. No.: 16/776,561

(22) Filed: Jan. 30, 2020

(51) Int. Cl. G16H 70/60; G06F 16/28; G16H 10/60; H04W 84/04; G06Q 50/26

(52) U.S. Cl. CPC G16H 70/60; G06F 16/285; G06Q 50/265; H04W 84/042; G16H 10/60

(57) ABSTRACT

A method for contacting a predetermined entity based on a death event classification includes: assigning a numeric value to a first partial score based on the method which caused the subject's death; assigning a numeric value to a second partial score based on the subject's history of mental illness; assigning a numeric value to a third partial score based on consistency of the death scene evidence with suicidal dynamics; assigning a numeric value to a fourth partial score based on the number of means that caused the subject's death; assigning a numeric value to a fifth partial score based on the compatibility of means and injuries with suicidal dynamics; summing the partial scores to obtain a total score; adding a correction factor if at least one suicide indicator is present; and classifying the death event. If the death event is classified as incompatible with suicide, the predetermined entity is notified.

### FIELD OF THE INVENTION

The present invention relates to the field of methods and systems for investigation of a death scene.

In particular, it relates to a method for the investigation of a death scene in order to identify a possible suicide or a homicide and act properly.

### BACKGROUND

Suicide is a serious global public health problem and the World Health Organization estimate that about 800,000 people die due to suicide every year (www.who.int/mental_health/suicide-prevention/en/). The number of suicide is higher than the homicide rates in many Western Countries. In Italy for example, the last annual data available record 468 homicides compared to 3935 suicides (www.istat.it/it/archivio/suicidi) and several studies suggest that the rate of suicides is underestimated (C. Katz, J. Bolton, J. Sareen, The prevalence rates of suicide are likely underestimated worldwide: why it matters, Soc. Psychiatry Psychiat.r Epidemiol. 51 (January (1)) (2016) 125-127).

The early correct framing of a case as suicide, as well as being useful for statistics and prevention strategies, is important for the medico-legal expert in order to arrive at the appropriate classification of the case from the beginning.

The distinction between death due to suicide, murder or accident has always been a subject of great interest in forensic medicine and the death scene investigation is critically important to identify the real dynamics of the facts.

The ambiguity of some scenarios, the complexity of the death scene and the range of information that is collected during the on-site inspection may mislead the forensic expert and lead to a vision of the event that can be strongly influenced by the preparation and the initial orientation of the medical examiner, particularly in cases of suicide that have uncommon features (D. Cusack, S. D. Ferrara, E. Keller, B. Ludes, P. Mangin, M. V\check{a}li, N. Vieira, European Council of Legal Medicine (ECLM) principles for on-site forensic and medico-legal scene and corpse investigation, Int. J. Leg. Med. 131 (July (4)) (2017) 1119-1122; C. A. J. van den Eeden, C. J. de Poot, P. J. van Koppen, Forensic expectations: investigating a crime scene with prior information, Sci. Justice 56 (December (6)) (2016) 475-481; J. Goodin, R. Hanzlick, Mind your manners. Part II: general results from the National Association of Medical Examiners Manner of Death Questionnaire, 1995, Am. J. Forensic Med. Pathol. 18 (September (3)) (1997) 224-227; R. Hanzlick, J. Goodin, Mind your manners. Part III: individual scenario results and discussion of the National Association of Medical Examiners Manner of Death Questionnaire, 1995, Am. J. Forensic Med. Pathol. 18 (September (3)) (1997) 228-245; T. H. Lu, S. M. Sun, S. M. Huang, J. J. Lin, Mind your manners: quality of manner of death certification among medical examiners and coroners in Taiwan, Am. J. Forensic Med. Pathol. 27 (December (4)) (2006) 352-354).

The standardization of this phase is therefore of crucial importance for the early identification of the dynamic of the facts.

To achieve a correct suicide diagnosis, as in each diagnostic path, it is important to consider and promptly identify both risk factors and characteristic findings of a self-induced death. Suicide risk factors had been identified, in the past years, mainly through several studies based on the psychological autopsy, which is the most direct technique currently available for examining the relationship between particular antecedents and suicide (J. T. Cavanagh, A. J. Carson, M. Sharpe, S. M. Lawrie, Psychological autopsy studies of suicide: a systematic review, Psychol. Med. 33 (April (3)) (2003) 395-405 Review. Erratum in: Psychol. Med. 2003 July; 33(5):947; E. T. Isometsa, Psychological autopsy studies—a review, Eur. Psychiatry 16 (November (7)) (2001) 379-385 Review), while findings characteristic of suicide, which allow the distinction from homicides, accidents or natural death, have been the object of a large number of studies, mainly focused on the means and injuries representative of suicidal dynamics.

Currently, in fact, there is a lack of specific international guidelines for the identification and consistent determination of suicide among medico-legal experts and coroners, even if the first operational criteria for the suicide determination date back to 1988 (J. L. Parai, N. Kreiger, G. Tomlinson, E. M. Adlaf, The validity of the certification of manner of death by Ontario coroners, Ann. Epidemiol. 16 (November (11)) (2006) 805-811 Epub 2006 Apr. 18. M. L. Rosenberg, L. E. Davidson, J. C. Smith, A. L. Berman, H. Buzbee, G. Gantner, G. A. Gay, B. Moore-Lewis, D. H. Mills, D. Murray, et al., Operational criteria for the determination of suicide, J. Forensic Sci. 33 (November (6)) (1988) 1445-1456). Several studies have shown that the agreement of forensic experts on the classification of controversial but representative death scenarios varies (J. Goodin, R. Hanzlick, Mind your manners. Part II: general results from the National Association of Medical Examiners Manner of Death Questionnaire, 1995, Am. J. Forensic Med. Pathol. 18 (September (3)) (1997) 224-227; R. Hanzlick, J. Goodin, Mind your manners. Part III: individual scenario results and discussion of the National Association of Medical Examiners Manner of Death Questionnaire, 1995, Am. J. Forensic Med. Pathol. 18 (September (3)) (1997) 228-245; T. H. Lu, S. M. Sun, S. M. Huang, J. J. Lin, Mind your manners: quality of manner of death certification among medical examiners and coroners in Taiwan, Am. J. Forensic Med. Pathol. 27 (December (4)) (2006) 352-354).

Furthermore, it has been demonstrated that prior information given to crime scene investigators influence their perception and interpretation of the death scene (C. A. J. van den Eeden, C. J. de Poot, P. J. van Koppen, Forensic expectations: investigating a crime scene with prior information, Sci. Justice 56 (December (6)) (2016) 475-481), which is, in fact, interpreted differently depending on how it is presented, and both the initial and the final assessment are influenced by the prior information given. Other studies have shown that, particularly for the diagnosis of suicide, there is too much emphasis on circumstantial data and on the presence of suicide notes (I. R. H. Rockett, E. D. Caine, H. S. Connery, G. D'Onofrio, D. J. Gunnell, T. R. Miller, K.

B. Nolte, M. S. Kaplan, N. D. Kapusta, C. L. Lilly, L. S. Nelson, S. L. Putnam, S. Stack, P. Varnik, L. R. Webster, H. Jia, Discerning suicide in drug intoxication deaths: paucity and primacy of suicide notes and psychiatric history, PLoS One 13 (January (1)) (2018)).

A previous study has focused on the possibility of identifying cases of “typical suicide” through an interpretative analysis during the on-site inspection (L. Massaro, Unusual suicide in Italy: criminological and medico-legal observations-a proposed definition of “atypical suicide” suitable for international application, J. Forensic Sci. 60 (May (3)) (2015) 790-800), proposing a method based on the investigation of five main areas and the use of a scoring system, aimed at optimizing the study of the “body found in”, particularly in cases of equivocal death (D. G. Denning, Y. Conwell, D. King, C. Cox, Method choice, intent, and gender in completed suicide, Suicide Life Threat. Behay. 30 (Fall (3)) (2000) 282-288 PubMed PMID:11079640).

This approach permits the conversion from a negative diagnosis, based on exclusion of reliable elements which might ascribe the death to murder or accident, to a positive diagnosis of suicide, within the range of parameters of scientific probability, based on the presence of elements which probably point to suicide.

The possibility of diagnosing suicide or homicide based on a standardized analysis of elements is very important for the correct initial framing of the death scene. Also this would allow even a person not expert in the field to immediately take the proper actions.

It is therefore desired a method of analysis of the death scene, which allows an objective framing of the case and the early identification of those cases probably attributable to self-induced death or to homicide.

### SUMMARY

It has now been found a scoring system for the correct framing of a case starting from the death scene investigation (DSI).

It is an object of the invention a method for contacting a predetermined entity based on a classification of a death event comprising the following steps of:

assigning a value comprised within a first predetermined numeric range to a first partial score based on the method which caused the death of a subject;

assigning a value comprised within a second predetermined numeric range to a second partial score based on the subject's personal history of mental illness;

assigning a value comprised within a third predetermined numeric range to a third partial score based on the consistency of the death scene evidence with suicidal dynamics;

assigning a value comprised within a fourth predetermined numeric range to a fourth partial score based on the number of means that caused the death of the subject;

assigning a value comprised within a fifth predetermined numeric range to a fifth partial score based on the compatibility of means and injuries with suicidal dynamics;

making a sum of the values of the partial scores of steps a)-e) to obtain a total score;

adding to the total score a correction factor if at least one positive indicator of suicide is present;

classifying the death event as follows: if the total score is less than or equal to a first threshold the death is classified as suicide; if the total score is greater than the first threshold and it is less than or equal to a second threshold the death is classified as atypical suicide; if the score is greater than the second threshold, than the death is classified as incompatible with suicide;

if the death event is classified as incompatible with suicide contacting the predetermined entity by sending to it a signal through a telecommunication system.

The method of the invention allowing the classification of death events into categories of “typical suicide”, “atypical suicide” (divided into slightly, moderately and strongly atypical) and “incompatible with suicide” has been found to be efficient in the identification of self-inflicted deaths and can be useful to perform an objective evaluation of the scene, without this being influenced by the prior information received.

This method is able to provide a reliable and objective way of recording the on-site inspection findings for the initial assessment of a death scene, giving an indicator of the probability that the case is a case of suicide or homicide.

In case the classification step provides as a result that the death event is not a suicide, an immediate contact with predetermined entities, such as local police or judicial authority, is established. This is particularly useful and advantageous in case the method is performed by a non-expert user allowing him/her to immediately take the proper action.

The method has been effective in the identification of suicides in a case series applied, the total score and the partial scores being both inversely proportional to the probability of facing a suicide case.

### BRIEF DESCRIPTION OF THE DRAWINGS

The following detailed description of the preferred embodiment of the present invention will be better understood when read in conjunction with the appended drawings. For the purpose of illustrating the invention, there are shown in the drawings embodiments, which are presently preferred. In the drawings:

FIG. 1. Histograms representing the distribution of the number of cases for each total scores (from 0 to 10) according to the dynamics of death (suicide, accidental death, homicide).

FIG. 2. Absolute and relative (within parenthesis) frequency of suicide (S), accidental death (A) and homicide (H) in the each category.

### DETAILED DESCRIPTION OF THE PREFERRED EMBODIMENTS

Certain terminology is used in the following description for convenience only and is not limiting. The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

The method is based on the assignment of a “partial score”, preferably from 0 to 2, to each of five areas, which are: (1) statistical frequency of the suicidal method adopted by the victim, (2) victim's personal history of mental illness, (3) consistency of the crime scene evidence with a suicidal dynamic, (4) number of means adopted by the victim, and (5) compatibility of means and injuries with suicidal dynamics; where typically 0 is assigned to the typical characteristics of a suicidal dynamic, 1 to slightly atypical characteristics, and 2 to atypical features.

A “correction factor” can be applied in case of presence of indicators of suicide risk.

To each case corresponds a “total score” given by the arithmetic sum of the partial scores and, eventually, the correction factor, which imply the inclusion within a “category” of probability of a case of suicide: typical suicide, atypical suicide or death incompatible with suicide.

In an embodiment, the partial scores are comprised between 0 and 2.

In particular, each partial score can be 0, 1 or 2. The correction factor is preferably −1.

In such embodiment, the death event is classified as follows: if the total score is comprised between 0 and 1 the death is classified as suicide; if the total score is comprised between 2 and 8 the death is classified as atypical suicide; if the score is comprised between 9 and 10, the death is classified as incompatible with suicide.

In an embodiment, the method of the invention comprises the following steps of:

assigning a value between 0 and 2 to a first partial score based on the method which caused the death of a subject, wherein optionally 0 is assigned to the first partial score if the method has a statistical frequency as suicidal method greater than a first predetermined value; 1 is assigned to the first partial score if the method has a statistical frequency as suicidal method between a second predetermined value and the first predetermined value; and 2 is assigned to the first partial score if the method has a statistical frequency as suicidal method less than the second predetermined value, the first predetermined value being greater than the second predetermined value;

assigning a value between 0 and 2 to a second partial score based on the subject's personal history of mental illness, wherein optionally 0 is assigned to the second partial score if the subject presented at least one disorder selected from schizophrenia, borderline or antisocial personality, mood disorders, drug addiction and alcoholism; 1 is assigned to the second partial score if there is the suspicion of substance abuse and if the subject has a history of mood disorders or former addiction; 2 is assigned to the second partial score in case of absence of the above-mentioned diseases or in the case of lack of information;

assigning a value between 0 and 2 to a third partial score based on the consistency of the death scene evidence with suicidal dynamics, wherein optionally 0 is assigned to the third partial score if all the following events are detected: discovery of the weapon or of elements necessary for the performance of the hypothetical suicide near the cadaver, detection of a suicide note or farewell message, presence of ordered personal effects of the victim and/or absence of signs of a struggle or of forced entry in enclosed places; 1 is assigned to the third partial score in case of presence of the weapon or the elements necessary for the implementation of the hypothetical suicide in the vicinity of the cadaver and at least one of the following: absence of disorder and presence of open windows and doors or open space; and 2 is assigned to the third partial score in all the other cases;

assigning a value between 0 and 2 to a fourth partial score based on the number of means that caused the death of the subject, wherein optionally 0 is assigned to the fourth partial score if only one suicidal method is adopted by the subject or in case of absence of injuries to which death could be attributed; 1 is assigned to the fourth partial score if two suicidal methods are adopted; and 2 is assigned to the fourth partial score in the case of adoption of more than two suicidal methods or in case of presence of bruising and excoriations on the cadaver not attributable to precipitation;

assigning a value between 0 and 2 to a fifth partial score based on the compatibility of means and injuries with suicidal dynamics, wherein optionally 0 is assigned to the fifth partial score if the injuries are typical for suicide; 1 is assigned to the fifth partial score if injuries are considered on average compatible with suicide; and 2 is assigned to the fifth partial score if injuries are poorly compatible with suicide;

making a sum of the values of the partial scores of steps a)-e) to obtain a total score;

adding to the total score a correction factor of −1 if at least one positive indicator of suicide is present;

classifying the death event as follows: if the total score is comprised between 0 and 1 the death is classified as suicide; if the total score is comprised between 2 and 8 the death is classified as atypical suicide; if the score is comprised between 9 and 10, the death is classified as incompatible with suicide;

starting a call to a predetermined entity if the death event is classified as incompatible with suicide.

In step a), the statistical frequency of the suicidal method adopted by the victim is evaluated.

The statistical frequency of the method adopted for suicide varies considerably from country to country and with the gender of the victim (D. G. Denning, Y. Conwell, D. King, C. Cox, Method choice, intent, and gender in completed suicide, Suicide Life Threat. Behay. 30 (Fall (3)) (2000) 282-288; K. Hawton, Sex and suicide. Gender differences in suicidal behaviour, Br. J. Psychiatry 177 (2000) 484-485). For those reasons, the method distinguishes on the basis of sex and of the country of origin, assigning a score between 0 and 2 on the basis of the statistical frequency of choice of method of committing suicide. Such statistical data can be easily obtained by the skilled person using online available databases.

The score is assigned as disclosed above according to the principle that the less often the type of dynamics and methods of suicide are statistically represented, the greater the characteristics of atypical suicide are. See Table 1 for an example of statistical frequencies in Italy.

In a preferred embodiment, the first predetermined value is 15% and the second predetermined value is 10%.

The percentage that determines the score may be revised over time in relation to the statistical variation in prevalence/incidence of suicidal method registered over the years in each specific country.

TABLE 1
First item-partial score corresponding to the method adopted by the
victim, based on its statistical frequency as a suicidal method.
Statistical frequency of the suicidal method adopted by the victim (Italy)

| Partial score | Method (male) | Method (female) |
|---|---|---|
| 0 | Frequency >15% | Frequency >15% |
| 1 | 10 < frequency ≤ 15% | 10 < frequency ≤ 15% |
| 2 | Others (frequency <10%) | Others (frequency <10% |

In step b), the victim's personal history of mental illness is evaluated. One of the most significant risk factors for suicide is the presence of psychiatric disorders (J. T. Cavanagh, A. J. Carson, M. Sharpe, S. M. Lawrie, Psychological autopsy studies of suicide: a systematic review, Psychol. Med. 33 (April (3)) (2003) 395-405 Review. Erratum in: Psychol Med. 2003 July; 33(5):947; E. T. Isometsa, Psychological autopsy studies—a review, Eur. Psychiatry 16 (November (7)) (2001) 379-385 Review; E. L. Gómez-Durán, M. A. Forti-Buratti, B. Gutierrez-Lopez, A. Belmonte-Ibáñez, C. Martin-Fumadó, Psychiatric disorders in cases of completed suicide in a hospital area in Spain between 2007 and 2010, Rev. Psiquiatr. Salud Ment. 9 (January-March (1)) (2016) 31-38; M. K. Nock, I. Hwang, N. A. Sampson, R. C. Kessler, Mental disorders, comorbidity and suicidal behavior. Results from the National Comorbidity Survey Replication, Mol. Psychiatry 15 (8) (2010) 868-876). The suicide risk among mental health patients is even 12 times grater than the general population (R. C. Evenson, J. B. Wood, E. A. Nuttall, D. W. Cho, Suicide rates among public mental health patients, Acta Psychiatr. Scand. 66 (1982) 254-264) and psychological autopsies established that more than 90% of completed suicides have suffered from co-morbid mental disorders [J. T. Cavanagh et al.; E. T. Isometsa et al.; D. Wasserman, Z. Rihmer, D. Rujescu, M. Sarchiapone, M. Sokolowski, D. Titelman, et al., The European Psychiatric Association (EPA) guidance on suicide treatment and prevention, Eur. Psychiatry 27 (2012) 129-141).

The diseases most frequently associated with suicide are mood disorders, such as depressive disorders and bipolar disorders, schizophrenia, drug addiction and alcoholism, sometimes associated with specific neurobiological abnormalities. Overall 30-90% of all suicides have suffered from mood disorders preceding the fatal act, with a strong association between major depression and suicide (Y. Conwell, P. R. Duberstein, C. Cox, J. H. Herrmann, N. T. Forbes, E. D. Caine, Relationships of age and axis I diagnoses in victims of completed suicide: a psychological autopsy study, Am. J. Psychiatry 153 (1996) 1001-1008. S. J. Blumenthal, Suicide. A guide to risk factors assessment and treatment of suicidal patients, Med. Clin. N. Am. 72 (1988) 937-971[22] A. D. Lesage, R. Boyer, F. Grunberg, C. Vanier, R. Morissette, C. Menard-Buteau, M. Loyer, Suicide and mental disorders: a case-control study of young men, Am. J. Psychiatry 151 (1994) 1063-1068; J. Angst, A. Gamma, M. Gastpar, J. P. Lepine, J. Mendlewicz, A. Tylee, Gender differences in depression: epidemiological findings from the European DEPRES I and II studies, Eur. Arch. Psychiatry Clin. Neurosci. 252 (2002) 201-209).

Also drug addiction and alcoholism lead to a high risk of suicide. Among alcoholics, the lifetime risk of suicide is about 10-15%. Depression and/or alcoholism were comorbid in 85% of suicides (M. Montisci, C. Terranova, R. Snenghi, S. D. Ferrara, Chronic hydrocephalus and alcohol abuse in a young male suicide, Am. J. Forensic Med. Pathol. 27 (December (4)) (2006) 320-323; M. Pompili, G. Serafini, M. Innamorati, G. Dominici, S. Ferracuti, G. D. Kotzalidis, G. Serra, P. Girardi, L. Janiri, R. Tatarelli, L. Sher, D. Lester, Suicidal behavior and alcohol abuse, Int. J. Environ. Res. Public Health 7 (April (4)) (2010) 1392-1431; C. Yuodelis-Flores, R. K. Ries, Addiction and suicide: a review, Am. J. Addict. 24 (2015) 98-104; B. Barraclough, J. Bunch, B. Nelson, P. Sainsbury, A hundred cases of suicide: clinical aspects, Br. J. Psychiatry 125 (1974) 355-373; Y. Conwell, P. R. Duberstein, C. Cox, J. H. Herrmann, N. T. Forbes, E. D. Caine, Relationships of age and axis I diagnoses in victims of completed suicide: a psychological autopsy study, Am. J. Psychiatry 153 (1996) 1001-1008; A. L. Beautrais, P. R. Joyce, R. T. Mulder, D. M. Fergusson, B. J. Deavoll, S. K. Nightingale, Prevalence and comorbidity of mental disorders in persons making serious suicide attempts: a case-control study, Am. J. Psychiatry 153 (1996) 1009-1014; Z. Rihmer, A. Rihmer, P. Dome, Suicidal behaviour in patients with mood disorders, Evid. Based Psychiatric Care 1 (2015) 19-26).

Schizophrenia and some personality disorders have also been demonstrated as risk factors for suicide (lifetime risk of suicide of 5% in schizophrenic and 10% in borderline and antisocial personality disorders (Z. Rihmer, A. Rihmer, P. Dome, Suicidal behaviour in patients with mood disorders, Evid. Based Psychiatric Care 1 (2015) 19-26. K. Hor, M. Taylor, Suicide and schizophrenia: a systematic review of rates and risk factors, J. Psychopharmacol. 24 (November (4 Suppl)) (2010) 81-90. B. A. Palmer, V. S. Pankratz, J. M. Bostwick, The lifetime risk of suicide in schizophrenia: a reexamination, Arch. Gen. Psychiatry 62 (2005) 247-253. J. Paris, H. Zweig-Frank, A 27-year followup of patients with borderline personality disorder, Compr. Psychiatry 42 (2001) 482-487. S. B. Quello, K. T. Brady, C. S. Sonne, Mood disorders and substance use disorder: a complex comorbidiy, Sci. Pract. Perspect. 3 (1) (2006) 13-21).

In an embodiment of the present method, a score 0 is assigned in cases of disorders like schizophrenia, borderline or antisocial personality, and in cases of mood disorders, drug addiction or alcoholism; a score of 1 when there is the suspicion of substance abuse and for those with a history of mood disorders or former addiction, as summarized in Table 2. The score of 2 is assigned in case of absence of the above-mentioned diseases or in the case of lack of information.

Mood disorders can be for example depression, bipolar disorders, major depressive disorder, seasonal affective disorder (SAD), bipolar I disorder (i.e. manic depression), bipolar II disorder (i.e. mania, hypomania), cyclothymic disorders, disruptive mood dysregulation disorder, persistent depressive disorder (i.e. dysthymic disorder or dysthymia), premenstrual dysphoric disorder (from DSM V).

TABLE 2
Second item-partial score corresponding to the
victim's personal history of mental illness.
2) Victim's personal history of mental illness

| Partial score | History of mental illness |
|---|---|
| 0 | At least one between: |
| | mood disorders (depression, bipolar disorder, etc.) |
| | drug addiction |
| | alcoholism |
| | personality disorders with high risk of suicide (borderline, |
| | antisocial) |
| | schizophrenia |
| 1 | At least one between: |
| | former drug/alcohol addiction |
| | history of mood disorders |
| | drug abuse |
| 2 | Exclusion from the score 1 and 2 or lack of information |

Step c) evaluates the consistency of the death scene evidence with suicidal dynamics.

Evidence collected at the death scene is one of the key points for the early identification of suicides.

Three eventualities are reported, sometimes essential in the reconstruction of a suicidal dynamic. The first is the discovery of the weapon or of elements necessary for the performance of the hypothetical suicide near the cadaver.

Such elements can be selected from the group consisting of: firearms, knives, empty pharmaceutical confections or substances used for poisoning, a chair or other raised element in case of complete hanging.

The second eventuality is the detection of a suicide note or farewell message, in which suicidal ideation is reported, or the victim apologizes for his action or the presence of ordered personal effects of the victim. Such personal effects can be close to the body or, in the case of drowning or precipitation, in the place where the victim is suspected to have put his idea into practice. The third eventuality is absence of signs of a struggle, or of forced entry in enclosed places.

Based on these considerations, partial scores of 0, 1 and 2 can be assigned, as summarized in Table 3.

TABLE 3
Third item-partial score corresponding to the consistency
of the crime scene evidence with a suicidal dynamic.
3) Consistency of the crime scene evidence with a suicidal dynamic

| Partial score | Findings |
|---|---|
| 0 | All of these |
| | the presence of the weapon or elements necessary for the |
| | performance of the hypothetical suicide around the corpse |
| | suicide note or farewell message or finding of the ordered |
| | personal effects of the victim |
| | absence of signs of a struggle (or burglary as regards |
| | enclosed places) |
| 1 | Presence of the weapon or the elements necessary for the |
| | implementation of the hypothetical suicide in the vicinity |
| | of the cadaver + at least one of the following: |
| | absence of disorder |
| | presence of open windows and doors (indoors) or open |
| | space |
| 2 | Exclusion from the score 0 and 1 |

Step d) evaluates the number of means.

The use of multiple means for committing suicide often increases the difficulties in differentiation between suicide and homicide. Many studies have labeled those cases with the name “complex suicide” (S. Demirci, K. H. Dogan, Z. Erkol, I. Deniz, A series of complex suicide, Am. J. Forensic Med. Pathol. 30 (2009) 152-154), that is consensually defined as the use of more than one method to induce death. According to statistical evaluations, up to 5% of all suicides can be classified as complex suicide.

Being the most common occurrence, the score 0 is assigned to cases where only one method is adopted or in case of absence of injuries to which death could be attributed. This last case occurs when the differential diagnosis are poisoning death. Score 1 is assigned when two suicidal methods are adopted and score 2 in the case of adoption of more than two methods or in case of presence of bruising and excoriations on the cadaver not attributable to precipitation (Table 4).

For suicidal method is intended a self-inflicted manner of death with evidence (either explicit or implicit) of intent to die (ie the act of intentionally causing one's own death).

Suicidal methods are for example hanging (suffocation), poisoning (overdose), firearms, falls.

TABLE 4
Fourth item-partial score corresponding to the number of means
4) Number of means

| Partial score | Number of means |
|---|---|
| 0 | absence of injuries to which death could be attributed |
| | one |
| 1 | two |
| 2 | >two |
| | abrasions, excoriations and contusions not attributable to |
| | precipitation |

Step e) evaluates the compatibility of means and injuries with suicidal dynamics.

In many cases of violent death the body injury pattern is critically important for the differential diagnosis between suicide, murder and accidental death. The proposed score identifies typical characteristics of a suicidal dynamic, differentiating them based on the methods adopted by the victim.

In an embodiment, score 0 indicates that the injuries are typical for suicide, value 1 indicates that injuries are considered on average compatible with suicide and 2 poorly compatible or not detectable.

In those methods where the injury pattern analysis does not usually help in the differential diagnosis during the on-site inspection, like rail crashes, car accidents, precipitation, or self-incineration, a score 2 is assigned.

Exemplary embodiments are disclosed in the following.

When the injuries are due to drowning, firearms, bladed weapons, hanging, smothering, poisoning or strangulation, scores can be assigned as disclosed in the following Table 5.

TABLE 5
Fifth item-partial score corresponding to the compatibility
of means and injuries with a suicidal dynamic.
5) Compatibility of means and injuries with a suicidal dynamic

| | |
|---|---|
| Partial score | Features |

Drowning

| | |
|---|---|
| 0 | At least one among: |
| | related injuries (e.g. wrist slashing) |
| | absence of injuries |
| | self-tied knots or weights |
| 1 | Association with ecchymosis and bruising or other not |
| | lethal injuries |
| 2 | Exclusion from the score 0 and 1 |

Firearms

| | |
|---|---|
| 0 | All of these: |
| | typical localization-short barrel weapons: mouth, temporal |
| | and precordial regions |
| | long-barreled weapons: chin and abdomen |
| | contact or close range gunshot wounds |
| | direction bottom-up |
| | use of the right hand in right-handed subjects, of the left |
| | hand in the left-handed |
| | presence of gunpowder residues and/or blood splashes on |
| | the victim's hand |
| 1 | Typical localization + at least one among: |
| | contact or close range gun shotgun wounds |
| | direction bottom-up |
| | use of the right hand in right-handed subjects, the left hand |
| | in the left-handed |
| 2 | Exclusion from the score 0 and 1 |

Bladed weapons

| | |
|---|---|
| 0 | All of these: |
| | typical localization (incised wounds: inner surface of wrists |
| | and forearms, neck; stab wounds: heart region, neck, |
| | abdomen) |
| | hesitation marks |
| | no damages to clothes |
| | parallel injuries |
| 1 | Typical localization + at least one among: |
| | hesitation marks |
| | no damages to clothes |
| | parallel injuries |
| 2 | At least one among: |
| | defensive wounds |
| | chop wounds |
| | no typical localization |
| | exclusion from the score 0 and 1 |

Hanging

| | |
|---|---|
| 0 | All of these: |
| | complete hanging |
| | oblique, discontinuous, excoriated, unevenly deep ligature |
| | furrows in the neck |
| | absence of other injuries (with the exception of wrist |
| | slashing) |
| 1 | Oblique, discontinuous, excoriated, unevenly deep groove |
| | in the neck + at least one between: |
| | incomplete hanging |
| | no petechiae |
| 2 | At least one among: |
| | other injuries |
| | not excoriated ligature furrows |
| | exclusion from the score 0 and 1 |

Smothering

| | |
|---|---|
| 0 | All of these: |
| | presence of objects suitable to cause a simultaneous forced |
| | occlusion of mouth and nose still on the body |
| | absence of injuries in the inner part of the cheeks and of |
| | the lips and absence of ecchymosis and excoriations of the |
| | skin on the nose and the mouth |
| | absence of injuries possibly due to struggle |
| 1 | Presence of objects suitable to cause a simultaneous forced |
| | occlusion of mouth and nose still on the body + At least |
| | one between: |
| 2 | injuries in the inner part of the cheeks and of the lips |
| | ecchymosis and excoriations of the skin on the nose and the |
| | mouth |
| | presence of injuries possibly due to struggle |
| | exclusion from the score 0 and 1 |

Poisoning

| | |
|---|---|
| 0 | association with wrist slashing or other suicidal method |
| 1 | absence of injuries |
| 2 | exclusion from the score 0 and 1 |

Strangulation

| | |
|---|---|
| 0 | All of these: |
| | horizontal, continuous, excoriated and equally deep ligature |
| | furrow |
| | knotting |
| | absence of other injuries (with the exception of wrist |
| | slashing) |
| 1 | All of these: |
| | horizontal, continuous, excoriated and equally deep ligature |
| | furrow |
| | multiple revolutions |
| 2 | At least one between: |
| | association with other injuries |
| | semicircular skin lacerations possibly attributable to |
| | fingernails and scratches on the neck |
| | exclusion from the score 0 and 1 |

In firearm suicides the parts of the body commonly affected are the mouth, the temple and the chest (precordial region) in case of short barrel weapons, while in the case of long-barreled weapons the preferred areas are the chin and the abdomen. The direction of the shot is commonly bottom-up, with the use of the dominant arm. In gunshots to the head right-handed subjects prefer the use of the right hand, and left-handed subjects the left. Gunshot inlet wounds are usually those of contact or close range and the presence of gunpowder residues on the victim's hand means that the victim was involved in the shooting, which is why it is often a crucial element for the medico-legal identification of suicide cases, such as the presence of blood splashes on the hand used for the shot.

Suicidal incised wounds are frequent in the inner surface of wrists and forearms (wrist slashing) or on the neck (throat cutting); while stab wounds are commonly in the region of the heart, neck or abdomen, preceded by the denuding of the part of the body affected and are frequently repeated, parallel and close to each other. Commonly, hesitation marks are present, thin and superficial, symmetrical with respect to the deeper injuries. Conversely, in cases of murder, injuries with defense injuries located on the upper arms, instinctively outstretched to protect vital parts. Chopping injuries are extremely rare in suicide, observed in alienated people and made by self-inflicted injuries on the top of the head.

Referring to deaths due to asphyxia, hanging is a typical method used by suicides. Oblique, discontinuous and unequally deep ligature furrows are the most important types of evidence, even if it is present in simulated hangings or cadaver suspension. In those cases, the differential diagnosis is based on the vitality characteristic of the injuries, particularly on the presence of hemorrhages, bruising in proximity of the ligature furrow. Suicide by self-strangulation, although not frequent, can cause important difficulties in the distinction from homicide. It presupposes a constriction of the neck that lasts beyond the loss of consciousness implying the use of method by the victim to prevent the release of the tourniquet (i.e. multiple revolutions or knotting). The ligature furrow in these cases is continuous, horizontal and equally deep around the perimeter of the neck, and in most cases it is the only finding detectable, while in cases of murder the victim often shows signs of a struggle, semicircular skin lacerations possibly attributable to fingernails and scratches on the neck, inflicted in an attempt to break free from the noose.

Smothering is rarely used as a suicidal method and mostly by individuals suffering from psychiatric diseases, who occlude the nose and mouth with objects crammed into the airway, or use a plastic bag to cover the head. A homicide dynamic is also rare in adults and, in those cases, external findings are usually ecchymosis and excoriation on the mouth and on the nose, due to the compression of the aggressor's hands directly or through other means. Significant in cases of direct suffocation could be injuries on the internal part of the lips and the cheeks, represented by bruises and small tears produced by the teeth.

Asphyxiation by drowning is a common method of suicide but, frequently, it is not easy to distinguish between a suicidal and accidental dynamic. The suicidal nature of death is suggested by the presence of associated lesions, such as wrist slashing, of self made ligature or use of weights. Particular importance in such cases is attributed to medical history and circumstantial data, such as the discovery of suggestive findings (i.e. farewell messages) and the results of the judicial inspection (i.e. clothes of the victim found neatly folded along the river). Murder cases are rare and they are usually due to the stunning of the victim caused through other forms of violence, resulting in injuries to the corpse.

In most cases of poisoning, there are no injuries detectable, but in some cases the association with other injuries attributable to self-inflicted methods, such as the presence of cut injuries on the volar surface of the wrists, is indicative of suicide.

In step f) a correction factor can be input.

The correction factor is preferably −1.

This correction factor is based on the result of the analysis of any changes in lifestyle or habits on the part of the subject prior to death.

In particular, said correction factor is assigned if at least one of the positive indicators of suicide disclosed in Table 6 is present.

TABLE 6
Correction factor of the total score, based on the presence
of positive indicators of suicide (i.e. risk factors)

| Correction factor | |
|---|---|
| Partial score | Positive indicators of suicide |
| 1 | at least one between: |
| | Isolating oneself from friends and/or family members |
| | Communicating to relatives or friends a conviction |
| | of the meaninglessness of life (hopeless life) |
| | Getting rid of personal items of sentimental value |
| | A sudden improvement in mood after a period of mood |
| | deflection |
| | Neglecting personal hygiene and physical appearance |
| | Purchasing or accumulating pharmaceutical drugs |
| | Purchasing or procuring firearms |
| | Sudden renewed interest or loss of interest in religion |
| | Neglecting hobbies or daily routines |
| | Making appointments with a doctor for slight or |
| | dubious ailments |
| | Resigning from one's job |
| | Sudden interruption of work |
| | Change in performance at school, university, or work |
| | Changes in sleep and appetite patterns |

In step g) the values inserted in the preceding steps a)-f) are summed obtaining a total score.

In step h), the death event is classified depending on the total score obtained in step g).

In a particular embodiment, when the score is between 2 and 8, the death event is classified as atypical suicide. It can be further classified in slightly atypical suicide if the score is comprised between 2 and 3, in moderately atypical suicide if the score is between 4 and 5, in strongly atypical suicide if the score is comprised between 6 and 8, as shown in the following Table 7.

TABLE 7
Total scores corresponding to each category.

| | |
|---|---|
| Total score | Category |

| 0-1 | Typical suicide | |
|---|---|---|
| 2-3 | Atypical suicide | Slightly |
| 4-5 | | Moderately |
| 6-7-8 | | Strongly |

| | |
|---|---|
| 9-10 | Death incompatible with suicide |

In step i) a predetermined entity is contacted if the death event has been classified in the previous step as incompatible with suicide.

In particular, the predetermined entity is contacted by means of a signal, which is sent through a telecommunication system.

The signal to the predetermined entity can be a data signal.

The signal to the predetermined entity can be an analog or digital signal.

The signal to the predetermined entity can be at least one of the following types: electrical, electromagnetic wave, optical, radio wave, light signal, audio signal. In particular, it can be a phone call or a phone message.

The telecommunication system can comprise a wireless communication network and/or wired communication network. The telecommunication system can include at least one of the following network types: computer network, a telephone network, Internet.

Said predetermined entity is usually an entity, which should be informed in case of a homicide. It can be for example local and/or national police and/or local and/or national judicial authority.

It is also an object of the invention a computer program for carrying out the method above disclosed.

In particular, the computer program comprises instructions, which, when a computer executes the program, cause the computer to carry out the method above disclosed.

A computer-readable data carrier having stored thereon said computer program is also within the scope of the invention.

A data processing device comprising a processor configured to perform the method above disclosed is a further object of the invention.

Said data processing device may be an electronic device, such as a computer, a mobile phone or a tablet, which comprises said computer-readable data carrier.

Said electronic device is also object of the invention.

In an exemplary embodiment, the data processing device may comprise an input interface by means of which an user can inserts answers regarding different items detected in the crime scene according to the method of the invention and a output interface by means of which the computer program stored in the data processing device provides to the user a numerical output indicative of suicide, murder or accidental death based on the inserted answers.

The data processing device is also able to feed itself with the data entered, implementing in turn the validation of the method by processing the entered data.

Further embodiments herein may be formed by supplementing an embodiment with one or more element from any one or more other embodiment herein, and/or substituting one or more element from one embodiment with one or more element from one or more other embodiment herein.

Examples—The following non-limiting examples are provided to illustrate particular embodiments. The embodiments throughout may be supplemented with one or more detail from one or more example below, and/or one or more element from an embodiment may be substituted with one or more detail from one or more example below.

The method proposed was retrospectively applied to 180 cases of suspicious death in which both death scene investigation and standard forensic autopsy were performed. The cases, randomly selected from the database of the Legal Medicine of Padua University were divided equally between suicides, homicides and accidental deaths (B. Karger, E. Billeb, E. Koops, B. Brinkmann, Autopsy features relevant for discrimination between suicidal and homicidal gunshot injuries, Int. J. Legal Med. 116 (October (5)) (2002) 273-278). The period examined was between 2001 and 2017, with the exclusion of those cases with ages inferior to 18 years old at the time of death.

In all 180 cases the death scene investigation report was analyzed, together with health records, the on-site external examination of the body and circumstantial data with preliminary statements from relatives and/or suspects to the police officers. The analysis was blindly conducted.

The results were then compared with the definite dynamic of occurrence of the facts, ascertained at the completion of the investigations. Quantitative variables (partial and total scores) were analyzed reporting the mean and the median, and compared between dynamic categories by Kruskall-Wallis test. Predictive ability of the score in forecasting suicides was analyzed by univariate logistic regression, and the result reported as odds-ratio with 95% confidence interval.

Results

Total Score

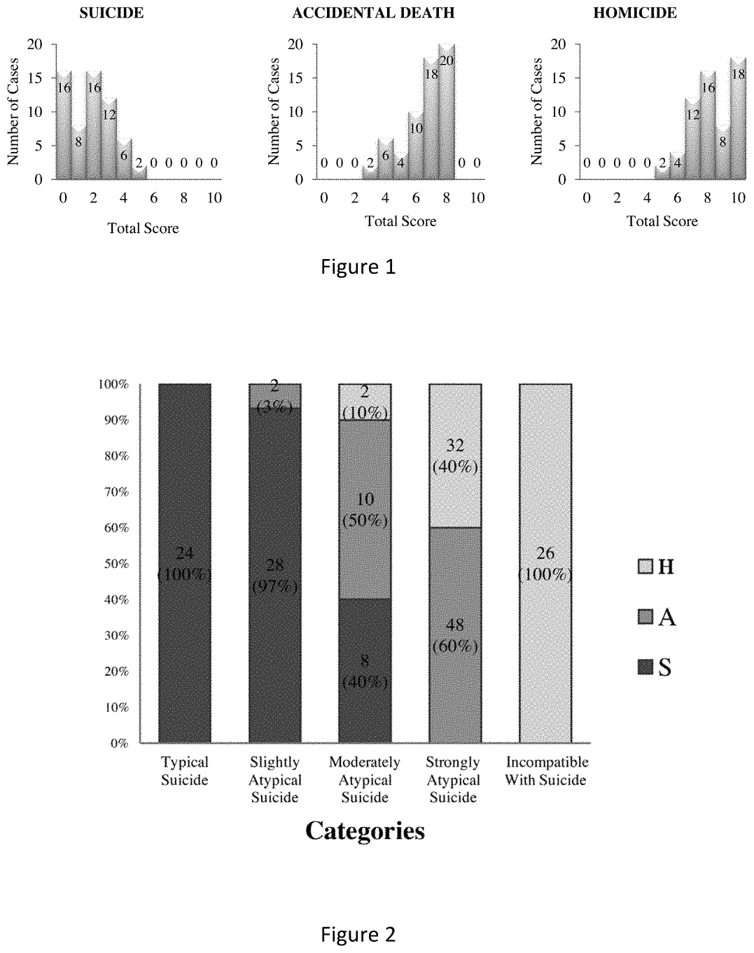

The results show a statistical correlation between the value of the total score and the probability of a suicidal dynamic (median: 2 suicides, 7 accidental deaths, 8 homicide), with a predominance of low scores in suicide cases, while in cases of accidental deaths, and even more in cases of homicide, scores lower than 3 are not registered and the main part obtain a score greater than or equal to 7 (FIG. 1). The increasing of every mark in the total score rises more than seven times the probability of a non-suicidal case, as shown by the Odds Ratio (7.41; IC 95% [2.28-24.02]).

Partial Score

Results show also a correlation between the value attributed to each item of the score and the probability of facing a suicide, as is reflected by the comparison among the means of the score attributed in each criteria, divided on the basis of the dynamic (Table 8), with the exception of the criteria “Number of means”, which assumes a partial score of 0 in all cases of accidental deaths.

TABLE 8
Mean of the partial scores given to each item, divided according
to the dynamic (suicide, accidental death, homicide).

| | Mean of the partial score | | | | |
|---|---|---|---|---|---|
| | Item 1 | Item 2 | Item 3 | Item 4 | Item 5 |
| Dynamic Suicide | 0.8 | 0.5 | 0.3 | 0.1 | 0.1 |
| Accidental death | 1.8 | 1.6 | 1.7 | 0 | 1.5 |
| Homicide | 1.8 | 1.8 | 1.9 | 0.9 | 1.8 |

Item 1—statistical frequency of the method adopted by the victim; item 2—victim's personal history of mental illness; item 3—consistency of the crime scene evidence with a suicidal dynamic; item 4—number of means; item 5—compatibility of means and injuries with a suicidal dynamic.

Categories

Concerning the subdivision into categories of the 180 cases analyzed, 24 has obtained a mark of 0 or 1, therefore belonging to the “typical suicide” category, while 26 cases have obtained higher marks of 9 or 10, therefore belonging to the “incompatible with suicide” category. In these cases the agreement of the final result with the effective dynamics was 100% as all 24 typical suicide corresponded to the suicide dynamics while all the 26 cases not compatible with suicide corresponded to homicide dynamics (FIG. 2).

The 130 cases that have reached an intermediate value, from 2 to 8, belong to the category of the “atypical suicide” and are subdivided as follows:

a. “slightly atypical suicide” (values 2 or 3): 28 suicides, 2 accidental deaths, 0 homicides;
b. “moderately atypical suicide” (values 4 or 5): 8 suicides, 10 accidental deaths, 2 homicides;
c. “strongly atypical suicide” (values 6, 7 or 8): 0 suicides, 48 accidental deaths, 32 homicides.

This scale aims to give a reliable and objective way of recording the on-site inspection findings for the initial assessment of a death scene, giving an indicator of the probability that the case is a case of suicide.

The score, in fact, was effective in the identification of suicides in the case series applied, the total score and the partial scores being both inversely proportional to the probability of facing a suicide case.

The exception of the partial score “number of means”, as resulting from the analysis of the median of the partial score (Table 8), is explained by the fact that an accidental death is generally caused by only one method. This feature, may allow the identification of cases that correspond, with high probability, to homicide dynamics, which are those that fall in the “incompatible with suicide” category (total score 9-10).

Based on the data collected, the best cut-off value to select for distinguishing a suicidal method from one that is not suicidal, with a high level of probability, is 4. In the cases that have been analysed, it can be seen that a value less than 4 represents 87% of suicide cases, compared to 3% of accidental deaths and 0% of homicide cases.

In cases where it is not possible to obtain the information already listed during the on-site inspection, the score can also be completed during the subsequent post-mortem investigations.

This study provides the first objective interpretative method of analysis of the death scene that, without expecting to reduce the complex death scene activities to the mere application of this method, and without aiming to replace all the necessary post-mortem ascertainments, can be used as a prognostic indicator of the likelihood of being faced with a case of suicide, while the higher the total score, the more difficult can be the management for the assessment of the manner of death and the inter-expert agreement.

The proposed score and the subsequent classification of suicides into categories of “typical suicide”, “atypical suicide” (divided into slightly, moderately and strongly atypical) and “incompatible with suicide” have been found to be efficient in the identification of self-inflicted deaths and can be useful to perform an objective evaluation of the scene, without this being influenced by the prior information received.

The references cited throughout this application are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

1. A method for contacting a predetermined entity based on a classification of a death event comprising the steps of:
a) assigning a value comprised within a first predetermined numeric range to a first partial score based on a method which caused the death of a subject;
b) assigning a value comprised within a second predetermined numeric range to a second partial score based on the subject's personal history of mental illness;
c) assigning a value comprised within a third predetermined numeric range to a third partial score based on a consistency of the death scene evidence with suicidal dynamics;
d) assigning a value comprised within a fourth predetermined numeric range to a fourth partial score based on the number of means that caused the death of the subject;
e) assigning a value comprised within a fifth predetermined numeric range to a fifth partial score based on a compatibility of means and injuries with suicidal dynamics;
f) summing the values of the partial scores of steps a)-e) to obtain a total score;
g) adding to the total score a correction factor if at least one positive indicator of suicide is present;
h) classifying the death event as follows: if the total score is less than or equal to a first threshold the death is classified as suicide; if the total score is greater than the first threshold and it is less than or equal to a second threshold the death is classified as atypical suicide; if the score is greater than the second threshold, than the death is classified as incompatible with suicide;
i) if the death event is classified as incompatible with suicide contacting the predetermined entity by sending a signal via a telecommunication system to the predetermined entity.

2. The method of claim 1 wherein in each step a)-e) said first, second, third, fourth and/or fifth predetermined numeric range is 0 to 2.

3. The method of claim 1 wherein in step a) 0 is assigned to the first partial score if the method has a statistical frequency as suicidal method greater than a first predetermined value; 1 is assigned to the first partial score if the method has a statistical frequency as suicidal method between a second predetermined value and the first predetermined value; and 2 is assigned to the first partial score if the method has a statistical frequency as suicidal method less than the second predetermined value, the first predetermined value being greater than the second predetermined value.

4. The method of claim 1 wherein in step b) 0 is assigned to the second partial score if the subject presented at least one disorder selected from schizophrenia, borderline or antisocial personality, mood disorders, drug addiction and alcoholism; 1 is assigned to the second partial score if there is the suspicion of substance abuse and if the subject has a history of mood disorders or former addiction; 2 is assigned to the second partial score in case of absence of the above-mentioned diseases or in the case of lack of information.

5. The method of claim 1 wherein in step c) 0 is assigned to the third partial score if all the following events are detected: discovery of the weapon or of elements necessary for the performance of the hypothetical suicide near the cadaver, detection of a suicide note or farewell message, presence of ordered personal effects of the victim and/or absence of signs of a struggle or of forced entry in enclosed places; 1 is assigned to the third partial score in case of presence of the weapon or the elements necessary for the implementation of the hypothetical suicide in the vicinity of the cadaver and at least one of the following: absence of disorder and presence of open windows and doors or open space; and 2 is assigned to the third partial score in all the other cases.

6. The method of claim 1 wherein in step d) 0 is assigned to the fourth partial score if only one suicidal method is adopted by the subject or in case of absence of injuries to which death could be attributed; 1 is assigned to the fourth partial score if two suicidal methods are adopted; and 2 is assigned to the fourth partial score in the case of adoption of more than two suicidal methods or in case of presence of bruising and excoriations on the cadaver not attributable to precipitation.

7. The method of claim 1 wherein in step e) 0 is assigned to the fifth partial score if the injuries are typical for suicide; 1 is assigned to the fifth partial score if injuries are considered on average compatible with suicide; and 2 is assigned to the fifth partial score if injuries are poorly compatible with suicide.

8. The method of claim 1 wherein in step g) said correction factor is −1.

9. The method of claim 1 wherein in step h) said first threshold is 1 and said second threshold is 8.

10. The method of claim 1 wherein in step i) said contacting a predetermined entity is starting a call to a predetermined entity.

11. The method of claim 4, wherein in step b) said mood disorder is depression, bipolar disorders, major depressive disorder, seasonal affective disorder (SAD), bipolar I disorder, bipolar II disorder, cyclothymic disorders, disruptive mood dysregulation disorder, persistent depressive disorder and/or premenstrual dysphoric disorder.

12. The method of claim 1 wherein in step e) the injuries are due to drowning and the scores are assigned as follows:
0 if it is present at least one among:
related injuries
absence of injuries and
self-tied knots or weights
1 if there is an association with ecchymosis and bruising or other not lethal injuries
2 for any case excluded from the scores 0 and 1; or
the injuries are due to firearms and the scores are assigned as follows:
0 if all of these are present:
typical localization
contact or close range gunshot wounds
direction bottom-up
use of the right hand in right-handed subjects, of the left hand in the left-handed
presence of gunpowder residues and/or blood splashes on the victim's hand
1 if typical localization and at least one among:
contact or close range gun shotgun wounds
direction bottom-up
use of the right hand in right-handed subjects, the left hand in the left-handed
2 for any case excluded from the scores 0 and 1; or
the injuries are due to bladed weapons and the scores are assigned as follows:
0 if all of these are present:
typical localization
hesitation marks
no damages to clothes
parallel injuries
1 if typical localization and at least one among:
hesitation marks
no damages to clothes
parallel injuries
2 if at least one is present among:
defensive wounds
chop wounds
no typical localization
exclusion from the score 0 and 1; or
the injuries are due to hanging and the scores are assigned as follows:
0 if all of these are present:
complete hanging
oblique, discontinuous, excoriated, unevenly deep ligature furrows in the neck
absence of other injuries with the exception of wrist slashing
1 if oblique, discontinuous, excoriated, unevenly deep groove in the neck is present and at least one between:
incomplete hanging
no petechiae
2 if at least one is present among:
other injuries
not excoriated ligature furrows
exclusion from the score 0 and 1; or
the injuries are due to smothering and the scores are assigned as follows:
0 if all of these are present:
presence of objects suitable to cause a simultaneous forced occlusion of mouth and nose still on the body
absence of injuries in the inner part of the cheeks and of the lips and absence of ecchymosis and excoriations of the skin on the nose and the mouth
absence of injuries possibly due to struggle
1 if presence of objects suitable to cause a simultaneous forced occlusion of mouth and nose still on the body and at least one between:
injuries in the inner part of the cheeks and of the lips
ecchymosis and excoriations of the skin on the nose and the mouth
2 if presence of injuries possibly due to struggle or exclusion from the score 0 and 1; or
the injuries are due to poisoning and the scores are assigned as follows:
0 if association with wrist slashing or other suicidal method
1 if absence of injuries
2 if exclusion from the score 0 and 1; or
the injuries are due to strangulation and the scores are assigned as follows:
0 if all of these are present
horizontal, continuous, excoriated and equally deep ligature furrow
knotting
absence of other injuries, with the exception of wrist slashing
1 if all of these are present:
horizontal, continuous, excoriated and equally deep ligature furrow
multiple revolutions
2 if at least one is present between:
association with other injuries
semicircular skin lacerations possibly attributable to fingernails and scratches on the neck
exclusion from the score 0 and 1.

13. The method of claim 1, wherein in step g) said positive indicator of suicide is one or more event selected from:
isolating from friends and/or family members;
communicating to relatives or friends a conviction of the meaninglessness of life (hopeless life);
getting rid of personal items of sentimental value;
a sudden improvement in mood after a period of mood deflection;
neglecting personal hygiene and physical appearance;
purchasing or accumulating pharmaceutical drugs;
purchasing or procuring firearms;
sudden renewed interest or loss of interest in religion;
neglecting hobbies or daily routines;
making appointments with a doctor for slight or dubious ailments;
resigning from job;
sudden interruption of work;
change in performance at school, university, or work;
changes in sleep and appetite patterns.

14. The method of claim 1, wherein in step h), the death event is further classified in slightly atypical suicide if the score is comprised between 2 and 3, in moderately atypical suicide if the score is between 4 and 5, in strongly atypical suicide if the score is comprised between 6 and 8.

15. The method of claim 1 comprising the following steps:
a) assigning a value between 0 and 2 to a first partial score based on the method which caused the death of a subject, wherein optionally 0 is assigned to the first partial score if the method has a statistical frequency as suicidal method greater than a first predetermined value; 1 is assigned to the first partial score if the method has a statistical frequency as suicidal method between a second predetermined value and the first predetermined value; and 2 is assigned to the first partial score if the method has a statistical frequency as suicidal method less than the second predetermined value, the first predetermined value being greater than the second predetermined value;
b) assigning a value between 0 and 2 to a second partial score based on the subject's personal history of mental illness, wherein optionally 0 is assigned to the second partial score if the subject presented at least one disorder selected from schizophrenia, borderline or antisocial personality, mood disorders, drug addiction and alcoholism; 1 is assigned to the second partial score if there is the suspicion of substance abuse and if the subject has a history of mood disorders or former addiction; 2 is assigned to the second partial score in case of absence of the above-mentioned diseases or in the case of lack of information;
c) assigning a value between 0 and 2 to a third partial score based on the consistency of the death scene evidence with suicidal dynamics, wherein optionally 0 is assigned to the third partial score if all the following events are detected: discovery of the weapon or of elements necessary for the performance of the hypothetical suicide near the cadaver, detection of a suicide note or farewell message, presence of ordered personal effects of the victim and/or absence of signs of a struggle or of forced entry in enclosed places; 1 is assigned to the third partial score in case of presence of the weapon or the elements necessary for the implementation of the hypothetical suicide in the vicinity of the cadaver and at least one of the following: absence of disorder and presence of open windows and doors or open space; and 2 is assigned to the third partial score in all the other cases;
d) assigning a value between 0 and 2 to a fourth partial score based on the number of means that caused the death of the subject, wherein optionally 0 is assigned to the fourth partial score if only one suicidal method is adopted by the subject or in case of absence of injuries to which death could be attributed; 1 is assigned to the fourth partial score if two suicidal methods are adopted; and 2 is assigned to the fourth partial score in the case of adoption of more than two suicidal methods or in case of presence of bruising and excoriations on the cadaver not attributable to precipitation;
e) assigning a value between 0 and 2 to a fifth partial score based on the compatibility of means and injuries with suicidal dynamics, wherein optionally 0 is assigned to the fifth partial score if the injuries are typical for suicide; 1 is assigned to the fifth partial score if injuries are considered on average compatible with suicide; and 2 is assigned to the fifth partial score if injuries are poorly compatible with suicide;
f) making a sum of the values of the partial scores of steps a)-e) to obtain a total score;
g) adding to the total score a correction factor of −1 if at least one positive indicator of suicide is present;
h) classifying the death event as follows: if the total score is comprised between 0 and 1 the death is classified as suicide; if the total score is comprised between 2 and 8 the death is classified as atypical suicide; if the score is comprised between 9 and 10, the death is classified as incompatible with suicide;
i) starting a call to a predetermined entity if the death event is classified as incompatible with suicide.

16. A computer program for carrying out the method of claim 1.

17. A computer-readable data carrier having stored thereon said computer program of claim 16.

18. A data processing device comprising a processor configured to perform the method of claim 1.

19. A data processing device comprising a processor configured to perform the method of claim 1, wherein the data processing device is a computer, a mobile phone or a tablet, which comprises a computer-readable data carrier having stored thereon a computer program for carrying out the method.
